(19) United States
(12) Patent Application Publication
(10) Pub. No.: US 2026/0262969 A1
(43) Pub. Date: Sep. 10, 2026

(54) DEVICE AND SYSTEM FOR PULSE OXIMETRY BASED ON BIOELECTRICAL IMPEDANCE

(71) Applicant: MONASH UNIVERSITY, CLAYTON (AU)

(72) Inventors: Mehmet Rasit YUCE, CLAYTON (AU); Fatemeh HEYDARI, CLAYTON (AU); Neil TOM, CLAYTON (AU); Hongqiang XU, CLAYTON (AU)

(21) Appl. No.: 19/162,871

(22) PCT Filed: Mar. 6, 2024
(86) PCT No.: PCT/AU2024/050183

§ 371 Date: Sep. 5, 2025

(30) Foreign Application Priority Data
Mar. 6, 2023 (AU) 2023900599

(51) Int. Cl. A61B 5/145; A61B 5/00; A61B 5/0245; A61B 5/0531

(52) U.S. Cl. CPC A61B 5/14542; A61B 5/0245; A61B 5/0531; A61B 5/6802; A61B 5/7225; A61B 5/7257; A61B 5/7278

(57) ABSTRACT

Embodiments relate generally to a method of determining blood oxygen saturation, comprising: receiving, at a computing device, a first bioimpedance (BImp) measurement signal of a patient of a first frequency, and a second bioimpedance measurement signal of a patient of a second frequency different from the first frequency; wherein, including, for each of the first and second BImp measurement signals, an arterial pulse wave representing impedance changes through an artery of the patient over time; filtering each of the first and second BImp measurement signals; performing, on the first and second filtered BImp measurement signals, feature point extraction to generate a plurality of arterial pulse wave features; determining phase shift information based on the plurality of arterial pulse wave features for each of the first and second BImp measurement signals; and determining, based on the determined phase shift information, a blood oxygen level and saturation percentage of the patient.

### TECHNICAL FIELD

Embodiments generally relate to devices, systems, and methods for measuring blood oxygen levels. In particular, embodiments relate to devices, systems, and methods for measuring oxygen saturation based on bioelectrical impedance.

### BACKGROUND

Oxygen is the root of life in humans and the density of oxygen within blood cells affects the performance of limbs. Lack of oxygen, known as hypoxia, can damage the brain and heart, and a large reduction of oxygen for more than a few minutes can be fatal. In a human with good health, the rate of oxygen in haemoglobin is about 1.34 ml per gram (the haemoglobin concentration in the blood is 15 g/dl, approximately every 100 ml of blood contains 20 ml of haemoglobins saturated with oxygen).

The arterial blood oxygen saturation, SaO2, is determined based on the percentage of the haemoglobin molecules saturated with oxygen (oxyhaemoglobin) in the arterial blood. The measurements of SaO2 range from 0% to 100%, and it varies from 94% to 100% in healthy adults. The most accurate method to measure SaO2 is invasive arterial blood gas analysis. This method is available in hospitals and is time-consuming. The alternative is pulse oximetry which uses photoplethysmography (PPG) signal. Pulse oximetry results in non-invasive practical measurement of arterial blood oxygen saturation (SaO2), referred to as peripheral oxygen saturation, SpO2, (the term SpO2 indicates the SaO2 measured by pulse oximetry).

Pulse oximetry is a non-invasive and commercially available technology that provides almost continuous readings and has achieved developing popularity in a wide variety of clinical applications over the last decades. In current day medical practice, pulse oximetry is essential and is the standard of healthcare in hospitals and intensive care. Other objectives of using pulse oximetry include anaesthesia, emergency medicine, sleep apnea monitoring, and postoperative recovery.

Pulse oximetry defines the density of oxyhaemoglobin (HbO2) and deoxyhaemoglobin (Hb) by emitting two PPG wavelengths to the skin. Pulse oximetry defines the density of oxyhaemoglobin (HbO2) and deoxyhaemoglobin (Hb) by emitting two PPG wavelengths (red (660 nm) and infrared (940 nm)) to the skin. The sensor calculates the amount of red and infrared light transmitted (to the skin) and received (from the skin) and then determines the amount absorbed. The bone, tissue, and venous blood absorb most of the light. However, the absorption amounts do not vary during small periods. Therefore, the only light-absorbing part that changes within a pulse period, is the arterial blood. Oxyhaemoglobin and deoxyhaemoglobin have different absorption levels for different light lengths (oxyhaemoglobin absorbs infrared light more than red light, and deoxyhaemoglobin absorbs red light more than infrared light). Using this knowledge and mathematical models, the amount of light (red and infrared) collected by the receiver determines the oxyhaemoglobin in the blood and the SPO2 values.

The coefficients of the mathematical model used to calculate the SpO2 level in pulse oximeters can only be extracted using a phase of big data collection (as the calibration phase). Therefore, pulse oximeters are required to be assessed by standard departments (for acceptable accuracy) before commercialising. This is mostly done by comparing the pulse oximetry SpO2 reading with reference values extracted from CO-oximeter devices.

Reflectance and/or transmission pulse oximetry is based on PPG sensors which are limited to extremities (finger, earlobe, or toe), and because of limb movement, these locations are often subjected to a lot of motion artefacts. Pulse oximetry assumes that the optical paths of the red and infrared lights are identical and similar. Since there is a difference between the paths of red and infrared light caused by penetration depths propertied of different wavelengths, this assumption is another challenge of pulse oximetry. Reflectance pulse oximetry may also be affected by venous pulsations. The pulsatility of arterial blood is being used to distinguish the other absorbers in the path of the lights from arterial blood absorbance (which is the part used to calculate SpO2). Venous pulsations may be observed in some cases and add error to the SpO2 estimation. This can be reduced by applying pressure onto the sensor, but make the device uncomfortable for long-term measurements.

It is desired to address or ameliorate one or more shortcomings or disadvantages of prior methods and devices for pulse oximetry measuring, such as reflectance and/or transmission pulse oximetry, or to at least provide a useful alternative thereto.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

Some embodiments relate to a method of determining blood oxygen saturation, the method may comprise: receiving, at a computing device, a first bioimpedance (BImp) measurement signal of a patient, wherein the first BImp measurement signal is of a first frequency; receiving, at a computing device, a second bioimpedance measurement signal of a patient, wherein the second BImp measurement signal is of a second frequency different from the first frequency; wherein, including, for each of the first and second BImp measurement signals, an arterial pulse wave representing impedance changes through an artery of the patient over time; filtering each of the first and second BImp measurement signals using a band-pass filter, wherein the band-pass filter defines a frequency band; performing, on the first and second filtered BImp measurement signals, feature point extraction to generate a plurality of arterial pulse wave features; selecting, from the plurality of arterial pulse wave features, at least one arterial pulse wave feature for each of the first and second BImp measurement signals; comparing the at least one selected feature of the first BImp measurement signal and the at least one selected feature of the second BImp signal to determine phase shift information, wherein the phase shift information represents a phase shift between the first and second BImp measurement signals; and determining, based on the determined phase shift information, a blood oxygen level and saturation percentage of the patient.

The first and second BImp measurement signals may be received from a sensing device worn by the patient. The first frequency may be in the range of about 5 KHz to about less than 50 KHz. The second frequency may be in the range of about more than or equal to 50 KHz to about 100 KHz.

The receiving may be performed continuously and wherein the steps of filtering, performing, selecting, comparing, and determining may be performed repeatedly while receiving occurs.

The frequency band of the band-pass filter may have a lower stopband frequency between about 0.5 Hz and about 0.95 Hz. The frequency band of the band-pass filter may have a higher stopband frequency between about 15 Hz and about 20 Hz.

The method may further comprise, determining, based on at least one of the first and second BImp measurement signals, a heart rate frequency of the patient. Determining the heart rate frequency may include one or more of: performing a Fast Fourier transform (FFT) of at least one of the first and second BImp measurement signals, wherein a maximum point of an output of the FFT corresponds to the heart rate frequency; or performing peak detection of at least one of the first and second BImp measurement signals, wherein peaks are identified and a time between the identified peaks is converted to the heart rate frequency.

The method may further comprise, adjusting the higher stopband frequency of the frequency band of the band-pass filter, wherein the adjustment is based on the determined heart rate frequency of the patient. The adjustment of the frequency band of the band-pass filter, the steps of filtering, performing, selecting, comparing, and determining may be repeated. In some embodiments, performing the method may diagnose a medical condition.

Some embodiments relate to a computing device for monitoring a blood condition, the computer device may comprise: processing circuitry; a memory accessible to the processing circuitry, the memory including a signal processing code module; a communications module accessible to the processing circuitry, wherein the signal processing code module may include instructions, executable by the processing circuitry, to process bioimpedance (BImp) measurement signals received via the communications module; and wherein the signal processing code module may further include instructions, executable by the processing circuitry, to perform the following: filter at least two BImp measurement signals of different frequencies using a band-pass filter, wherein the band-pass filter defines a frequency band; perform feature point extraction on the at least two filtered BImp measurement signals to generate a plurality of arterial pulse wave features; select, from the plurality of arterial pulse wave features, at least one arterial pulse wave feature for each of the at least two BImp measurement signals; compare the at least one selected feature of each of the at least two BImp measurement signals to determine phase shift information, wherein the phase shift information represents a phase shift between the at least two BImp measurement signals; and determine, based on the determined phase shift information, an oxygen saturation percentage of the patient.

The at least two BImp measurement signals may be of different frequencies. The signal processing code module may further include instructions to determine, based on at least one of the at least two transformed BImp measurement signals, a heart rate frequency of the patient. The instructions of the signal processing code module may be performed continuously.

The signal processing code module may further include instructions to adjust the frequency band of the band-pass filter, wherein the adjustment is based on the determined heart rate frequency of the patient. The frequency band of the band-pass filter may have a lower stopband frequency between about 0.5 Hz and about 0.95 Hz and a higher stopband frequency between about 15 Hz and about 20 Hz.

Some embodiments relate to a system, the system may include the aforementioned computing device, and may further include, a wearable device, wherein the wearable device may comprise: processing circuitry; a memory accessible to the processing circuitry, the memory including a measurement code module; a communications module accessible to the processing circuitry, wherein the measurement code module may include instructions, executable by the processing circuit, to transmit bioimpedance (BImp) measurement signals to the computing device via the communications module; at least two electrodes attachable to skin of a person; and wherein the measurement code module may further include instructions, executable by the processing circuitry, to perform the following: output, via at least one of the at least two electrodes, at least two electrical stimulation signals to a patient, wherein the at least two electrical stimulation signals are of different frequencies; detect, via at least one of the at least two electrodes, at least two BImp measurement signals, wherein the at least two BImp measurement signals are based on the at least two electrical stimulation signals; and transmit, via the communications module, the at least two detected BImp measurement signals to the computing device.

Some embodiments relate to a kit, the kit may include the aforementioned computing device, and may further include a wearable device, wherein the wearable device may comprise: processing circuitry; a memory accessible to the processing circuitry, the memory including a measurement code module; a communications module accessible to the processing circuitry, wherein the measurement code module may include instructions, executable by the processing circuit, to transmit bioimpedance (BImp) measurement signals; at least two electrodes attachable to skin of a person; and wherein the measurement code module may further include instructions, executable by the processing circuitry, to perform the following: output, via at least one of the at least two electrodes, at least two electrical stimulation signals to a patient, wherein the at least two electrical stimulation signals are of different frequencies; detect, via at least one of the at least two electrodes, at least two BImp measurement signals, wherein the at least two BImp measurement signals are based on the at least two electrical stimulation signals; transmit, via the communications module, the at least two detected BImp measurement signals to the computing device; and wherein the computing device is configured to receive BImp measurement signals from the wearable device.

### BRIEF DESCRIPTION OF DRAWINGS

Embodiments are described in further detail below, by way of example and with reference to the accompanying drawings, in which:

FIG. 1 shows a schematic diagram of an example bioelectrical impedance measurement device, according to some embodiments;

FIG. 2 shows a process flow diagram of a method of determining blood oxygen saturation in a patient, according to some embodiments;

FIG. 3 shows an example graph of a plurality of filtered bioelectrical impedance measurement signals, according to some embodiments;

FIG. 4 shows an example graph of a plurality of filtered bioelectrical impedance measurement signals in the time-domain, according to some embodiments;

FIGS. 5A and 5B show example graphs illustrating the relationship between phase shift and blood oxygen saturation, according to some embodiments;

FIG. 6 shows a block diagram of a bioelectrical impedance circuit, according to some embodiments;

FIG. 7 shows a block diagram of a bioelectrical impedance signal generator and processing circuit of FIG. 6, according to some embodiments;

FIG. 8 shows a block diagram of an alternate bioelectrical impedance circuit, according to some embodiments;

FIG. 9 shows a block diagram of a bioelectrical impedance signal generator and processing circuit of FIG. 7, according to some embodiments;

FIG. 10 is a schematic diagram showing measurement of a signal injected through an artery of a patient, according to some embodiments;

FIG. 11 is an example frequency switching timing diagram, according to some embodiments;

FIG. 12 shows an example graph illustrating linear correlation of data of FIG. 5A, according to some embodiments; and

FIG. 13 shows a Bland-Altman plot illustrating accuracy of a mathematical model described herein, according to some embodiments.

### DESCRIPTION OF EMBODIMENTS

Embodiments generally relate to devices, systems, and methods for measuring blood oxygen levels. Particular embodiments relate to devices, systems, and methods for measuring oxygen saturation based on bioelectrical impedance.

Electrical properties of blood can vary due to the amount of oxygen carrying haemoglobin within the blood. Bioelectrical impedance measurements can be used to capture these changes in oxygenation of haemoglobin, and subsequently blood oxygen levels may be determined. Bioelectrical impedance of human tissue varies dependent on the frequency of the bioelectrical signals injected into the tissue. Further, properties of blood also varies dependent on the frequency of the bioelectrical signals injected. As a result, employing a combination of different frequency bioelectrical impedance signals allows measurements to have an increased sensitivity to changes in blood volume and content by analysing the different impedance variations at different frequencies. Electrical properties of blood and surrounding tissue may also be better characterised by a combination of different frequencies when compared to a single frequency.

Throughout the specification the term ‘patient’ will not be limited to an individual suffering from a condition, however, will be understood to mean any individual for whom it is desired to measure, assess, and/or track blood related information.

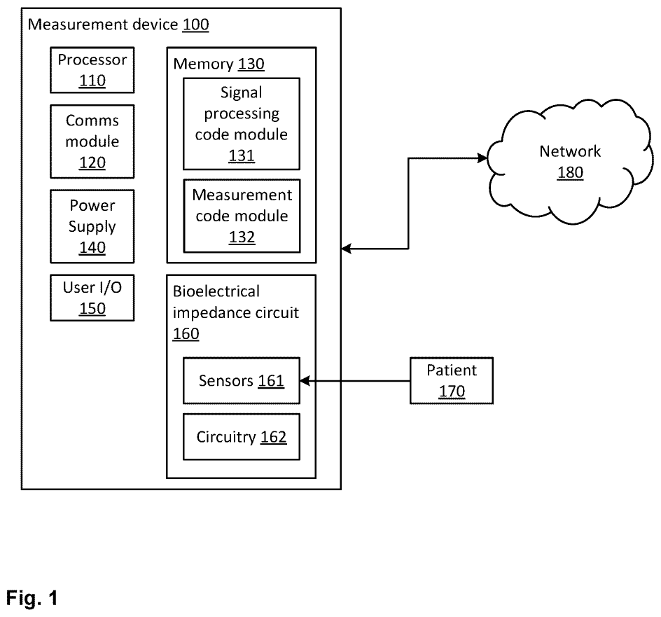

Referring to the drawings, FIG. 1 shows a schematic illustration of bioelectrical impedance (BImp) measurement device 100 (measurement device 100) for measuring parameters that can be used to determine a patient's oxygen saturation level (SpO2), blood oxygen level, heart rate, or blood pressure, according to some embodiments. Measuring device 100 is configured to measure parameters of a patient that are relatively dynamic, such as SpO2, blood oxygen level, heart rate, and/or blood pressure. These features are dynamic in that they commonly change relatively quickly over time in comparison to relatively static features, such as blood glucose, cholesterol, and fat levels.

In some embodiments, measurement device 100 comprises a processor 110 and a memory 130 accessible to processor 110. Processor 110 may be configured to access data stored in memory 130, to execute instructions stored in memory 130, and to read and write data to and from memory 130. Processor 110 may comprise one or more microprocessors, microcontrollers, central processing units (CPUs), application specific instruction set processors (ASIPs), or other processor capable of reading and executing instruction code.

Memory 130 may comprise one or more volatile or non-volatile memory types, such as RAM, ROM, EEPROM, or flash, for example. Memory 130 may be configured to store executable applications for execution by processor 110. For example, memory 130 may store signal processing code module 131 configured to determine blood oxygen saturation based on received bioelectrical impedance signals.

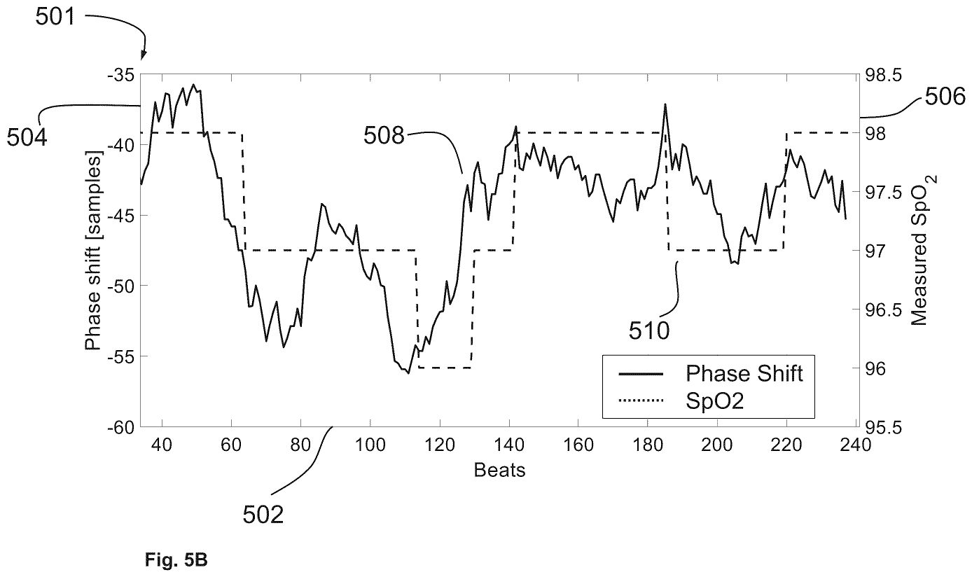

Memory 130 may also store measurement code module 132, which is described in further detail below in relation to FIGS. 6 to 9. Memory 130 may be configured to store blood measurement data, such as BImp measurement signals, blood oxygen levels, and/or blood pressure, for example.

To facilitate communication with external and/or remote devices, measurement device 100 further comprises a communications module 120.

Communications module 120 may allow for wired and/or wireless communication between measurement device 100 and external computing devices and components. Communications module 120 may facilitate communication via Bluetooth, USB, Wi-Fi, Ethernet, or via a telecommunications network, for example. According to some embodiments, communication module 120 may facilitate communication with external devices and systems via a network 180.

Network 180 may comprise one or more local area networks or wide area networks that facilitate communication between measurement device 100 and external computing devices. For example, according to some embodiments, network 180 may be the internet. However, network 180 may comprise at least a portion of any one or more networks having one or more nodes that transmit, receive, forward, generate, buffer, store, route, switch, process, or a combination thereof, etc. one or more messages, packets, signals, some combination thereof, or so forth. Network 180 may include, for example, one or more of: a wireless network, a wired network, an internet, an intranet, a public network, a packet-switched network, a circuit-switched network, an ad hoc network, an infrastructure network, a public-switched telephone network (PSTN), a cable network, a cellular network, a satellite network, a fibre-optic network, or some combination thereof.

Measurement device 100 further comprises a power supply 140 to provide electrical power to the various components of measurement device 100. In some embodiments, power supply 140 may be in the form of a rechargeable battery, for example, a nickel-metal hydride battery, a lithium-ion battery, a lead-acid battery, or a nickel-cadmium battery. To recharge the rechargeable battery, power supply 140 may further comprise a power port for connection to an external power source. In some embodiments, power supply 130 may be in the form of a non-rechargeable battery or a direct connection to an external power source, for example.

Measurement device 100 further comprises user input and output (I/O) 150 to allow communication between measurement device 100 and a user. User I/O 150 may comprise one or more of a camera, a speaker, buttons, sliders, a screen, and LEDs. In some embodiments, user I/O 150 may be used to alert the user of a particular event, such as the device being ready for use or the measurement process being complete, for example. In some embodiments, user I/O 150 may be used to provide visual representations of data to the user, for example.

In some embodiments, measurement device 100 further comprises a BImp circuit 160 to generate, output, process and detect electrical signals. In some embodiments, BImp circuit 160 may be an external device in communication with measurement device 100. BImp circuit 160 comprises at least two sensors 161 in electrical communication with measurement device 100. In some embodiments, BImp circuit 160 may comprise four sensors 161, for example. In some embodiments, BImp circuit 160 may comprise eight sensors 161, for example. In some embodiments, at least one of the two sensors 161 may function as an output of measurement device 100. In some embodiments, at least one of the sensors 161 may function as an input to measurement device 100. The sensors 161 may be electrodes, for example. The sensors 161 are configured to be disposed on the skin of a patient 170 in close proximity to each other. That is, the sensors 161 may be placed on the skin of a patient 170 such that they are approximately on the same part of the patient's 170 anatomy, for example. In some embodiments, the sensors 161 may be located on the wrist of the patient 170. In some embodiments, the sensors 161 may be located on at least one shoulder of the patient 170. BImp circuit 160 further comprises circuitry 162 to generate and process electrical signals input and output via sensors 161, as described below in relation to FIG. 6.

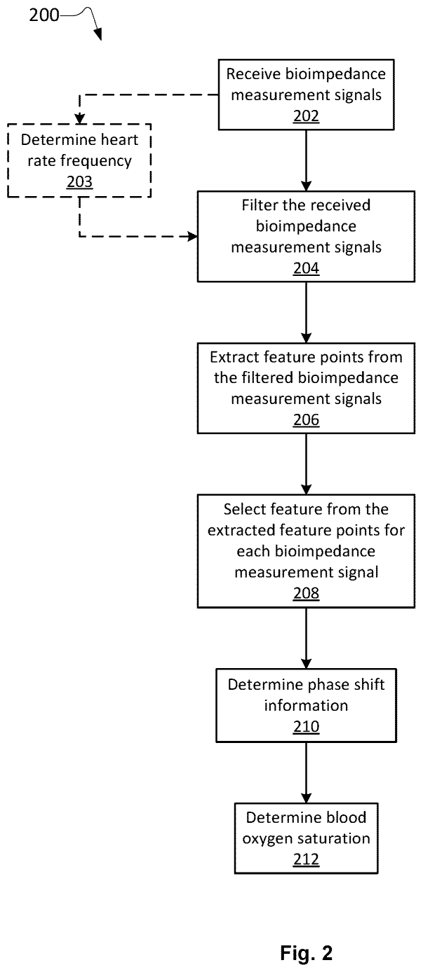

FIG. 2 is a process flow diagram of a method 200 of determining a patient's blood oxygen levels, according to some embodiments. In some embodiments, processor 110 may perform method 200 when executing signal processing code module 131. At step 202 of method 200, measurement device 100 receives a first BImp measurement signal and a second BImp measurement signal. Each of the first BImp measurement signal and the second BImp measurement signal comprise data measured over a time period, hereinafter referred to as the measuring time period. In some embodiments, each BImp measurement signals may be received from BImp circuit 160. In some embodiments, the BImp measurement signals may be retrieved from memory 130. The BImp measurement signals are received, or retrieved, in real-time, or near real-time. That is, the BImp measurement signals are received, or retrieved, in a manner that is unnoticeable to the user, such that the information is perceived to be received immediately.

The first BImp measurement signal is of a first frequency and the second BImp measurement signal is of a second frequency, wherein the second frequency is different from the first frequency. In some embodiments, the first frequency may be between about 5 KHz to about less than 50 KHz. The first frequency may be about 11 KHz, for example. In some embodiments, the second frequency may be between about more than or equal to 50 KHz to about 100 KHz. The second frequency may be about 71 KHz, for example. In some embodiments, measurement device 100 further receives a third BImp measurement signal of a third frequency, wherein the third frequency is different from the first and second frequencies. The third frequency may be between about 5 KHz and about 100 KHz, for example. In some embodiments, measurement device 100 further receives more than three BImp measurement signals.

In some embodiments, each BImp measurement signal comprises an arterial pulse wave, wherein the arterial pulse wave represents impedance changes through an artery of the patient 170 over time. That is, each BImp measurement signal is a measure of the changing impedance of haemoglobin within the artery of the patient 170 over time, for example. In some embodiments, the arterial pulse wave represents impedance changes through a vascular bed of the patient 170 over time. That is, each BImp measurement signal is a measure of the changing impedance of haemoglobin within a vascular bed of the patient 170 over time, for example. For the present disclosure, a vascular bed is a network of blood vessels, including arteries, veins, capillaries, arterioles, and venules, that facilitate blood circulation within an area or organ of the body. The body may therefore have multiple different vascular beds.

Measured impedance of haemoglobin may depend on the oxygenation of the haemoglobin itself. That is, oxyhaemoglobin may have a lower measured impedance and deoxyhaemoglobin may have a higher measured impedance, for example.

At step 204, each of the received first BImp measurement signal and the second BImp measurement signal are filtered using a band-pass filter. That is, the received signals are filtered to remove low frequency and high frequency noises. The band-pass filter defines a frequency band, where the frequency band has a lower stopband frequency and a higher stopband frequency. In some embodiments, the lower stopband frequency may be between about 0.5 Hz to about 0.95 Hz, for example. In some embodiments, the higher stopband frequency may be between about 50 KHz to about 100 KHz, for example. In some embodiments, the band-pass filter may be a Chebyshev type II band-pass filter. In some embodiments, the received first, second, and third BImp measurement signals are filtered using the band-pass filter. In some embodiments, each received BImp measurement signal is filtered using the band-pass filter.

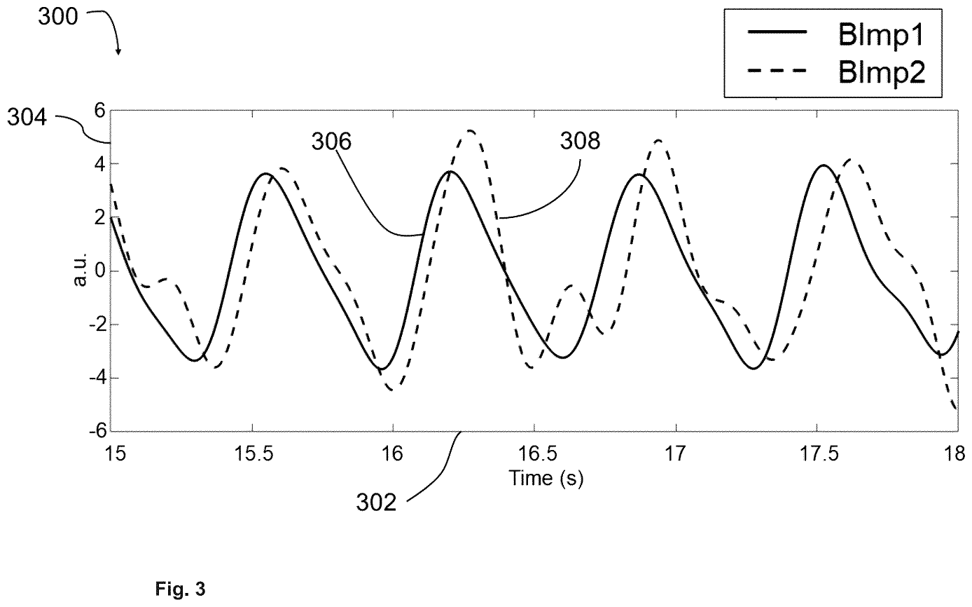

FIG. 3 shows an example graph 300 of a portion of the measuring time period of a filtered first BImp measurement signal 306 and a filtered second BImp measurement signal 308, according to some embodiments. The x-axis 302 of the graph 300 represents samples of the measurements. That is, for each filtered BImp measurement signal 306 and 308, the x-axis 302 represents a plurality of measurements of impedance changes through an artery, or vascular bed, of the patient 170 over time, for example. The y-axis 304 represents impedance magnitude for each measured sample in arbitrary units. In the example graph 300, the filtered first BImp measurement signal was measured at an injected current frequency of 11 KHz and the filtered second BImp measurement signal was measured at an injected current frequency of 71 KHz.

In some embodiments, method 200 may comprise step 203. Step 203 may occur immediately following step 202 and prior to step 204. At step 203, the heart rate frequency (HRF) of the patient 170 is determined. In some embodiments, to determine the HRF of the patient 170, a frequency domain approach, such as a Fast Fourier transform (FFT), is performed on at least one of the received BImp measurement signals. That is, a FFT process is performed on either the first BImp measurement signal or the second BImp measurement signal. In some embodiments, the FFT process may be performed on one of the first, second, or third BImp measurement signals. In some embodiments, the FFT process may be performed on one or more the received BImp measurement signals. Performing FFT on the at least one BImp measurement signal results in a frequency domain representation of the BImp measurement signal. The maximum point of the resulting frequency domain representation corresponds to the HRF of the patient 170. In some embodiments, the higher stopband frequency of the band-pass filter may be dynamically adjusted based on the determined HRF using a FFT. That is, the higher stopband frequency may be adjusted to a value equal to the HRF plus an additional about 3 Hz.

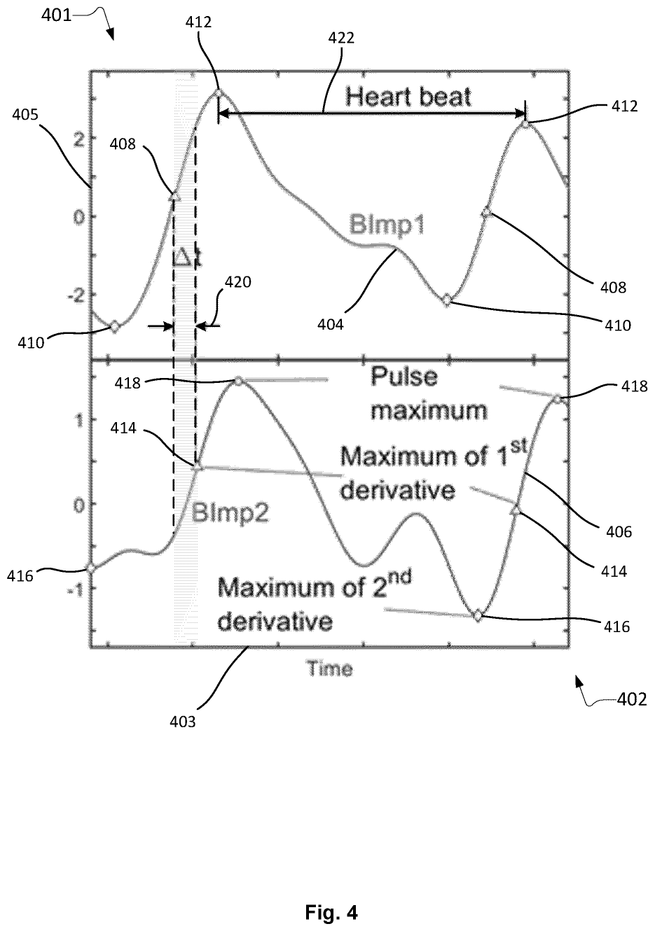

At step 206, feature point extraction is performed on each of the first and second BImp measurement signals to generate a plurality of arterial pulse wave features. In some embodiments, feature point extraction is performed on the first, second, and third BImp measurement signals to generate the plurality of arterial pulse wave features. In some embodiments, feature point extraction is performed on each of the BImp measurement signals to generate the plurality of arterial pulse wave features. That is, a plurality of arterial pulse wave features are extracted from each of the filtered first and second BImp measurement signals, as shown in FIG. 4, for example.

FIG. 4 shows an example graph 401 of a portion of the measuring time period of the filtered first BImp measurement signal represented in the time-domain 404, according to some embodiments. FIG. 4 further shows an example graph 402 of a portion of the measuring time period of the second BImp measurement signal represented in the time domain 406, according to some embodiments. Each graph 401 and 402 has an x-axis 403 representing time, and a y-axis 405 representing impedance magnitude for each measured sample in arbitrary units.

In performing feature point extraction, processor 110 performing method 200, determines 1^{st} and 2^{nd} derivative functions for the first BImp measurement signal and 1^{st} and 2^{nd} derivative functions for the second BImp measurement signal over the measuring time period. The processor 110 then determines a plurality of 1^{st} derivative maximums 408 of the 1^{st} derivative function of the first BImp measurement signal 404 and a plurality of 2^{nd} derivative maximums 410 of the 2^{nd} derivative function of the first BImp measurement signal 404 over the measuring time period. The processor 110 then determines a plurality of 1^{st} derivative maximums 414 of the 1^{st} derivative function of the second BImp measurement signal 406 and a plurality of 2^{nd} derivative maximums 416 of the 2^{nd} derivative function of the second BImp measurement signal 406 over the measuring time period.

In some embodiments, the plurality of 2^{nd} derivative maximums 410 of the first BImp measurement signal 404 and the plurality of 2^{nd} derivative maximums 416 of the second BImp measurement signal may be used to determine hemodynamic information. The plurality of 2^{nd} derivative maximums 410 and the plurality of 2^{nd} derivative maximums 416 may be used to determine timing and a shape of a dicrotic notch of the first and second BImp measurement signals 404 and 406, respectively.

In some embodiments, the processor 110 may also determine a plurality of first pulse maximums 412 of the first BImp measurement signal 404 over the measuring time period. That is, the processor 110 determines the peak points of the waveform of the first BImp measurement signal 404 over the measuring time period to be the first pulse maximums 412, for example. The processor 110 may then also determine a plurality of second pulse maximums 418 of the second BImp measurement signal 406 over the measuring time period. That is, the processors 110 determines the peak points of the waveform of the second BImp measurement signal 406 over the measuring time period to be the second pulse maximums 418, for example. In some embodiments, the waveform portion between each pulse maximum 412 point represents a single heartbeat 422 of the patient 170. In some embodiments, the waveform portion between each second pulse maximum 418 point represents a single heartbeat 422 of the patient 170. Each of the first and second BImp measurement signals may comprise a plurality of heartbeats (not shown) over the measuring time period.

Each determined heartbeat 422 of the first BImp measurement signal 404 comprises a singular 1^{st} derivative maximum 408 and a singular 2^{nd} derivative maximum 410. Each determined heartbeat 422 of the second BImp measurement signal 406 comprises a singular 1^{st} derivative maximum 414 and a singular 2^{nd} derivative maximum 416. That is, each heartbeat 422 of the plurality of heartbeats over the measuring time period contain a 1^{st} derivative maximum 408 and 414 and a 2^{nd} derivative maximum 410 and 416, for example.

In some embodiments, in performing feature point extraction, processor 110 further determines 1^{st} and 2^{nd} derivative functions of the third BImp measurement signal over the measuring time period. The processor 110 then further determines a plurality of 1^{st} derivative maximums of the 1^{st} derivative function of the third BImp measurement signal and a plurality of 2^{nd} derivative maximums of the 2^{nd} derivative function of the third BImp measurement signal over the measuring time period. In some embodiments, processor 110 determines 1^{st} and 2^{nd} derivative functions of each BImp measurement signal over the measurement period and respective 1^{st} and 2^{nd} derivative maximums.

In some embodiments, the plurality of 2^{nd} derivative maximums of the third BImp measurement signal may be used to determine hemodynamic information. The plurality of 2^{nd} derivative maximums 410, the plurality of 2^{nd} derivative maximums 416, and/or the plurality of 2^{nd} derivative maximums of the third BImp measurement signal may be used to determine timing and a shape of a dicrotic notch of the first (404), second (406), and/or third BImp measurement signals, respectively.

In some embodiments, the processor 110 may also determine a plurality of third pulse maximums of the third BImp measurement signal over the measuring time period. That is, the processor 110 determines the peak points of the waveform of the third BImp measurement signal over the measuring time period to be the third pulse maximums, for example. In some embodiments, the waveform portion between each third pulse maximum point represents a single heartbeat of the patient 170. The third BImp measurement signal may comprise a plurality of heartbeats over the measuring time period. In some embodiments, processor 110 determines a plurality of pulse maximums for each BImp measurement signal over the measuring time period.

Each determined heartbeat of the third BImp measurement signal comprises a singular 1^{st} derivative maximum of the third BImp measurement signal and a singular 2^{nd} derivative maximum of the third BImp measurement signal. That is, each heartbeat of the plurality of heartbeats over the measuring time period contain a 1^{st} derivative maximum of the third BImp measurement signal and a 2^{nd} derivative maximum of the third BImp measurement signal, for example.

In some embodiments, to determine the HRF of the patient 170, an individual or combination time domain approach, such as peak detection, is performed on at least one of the received BImp measurement signals. Peak detection utilises the determined first pulse maximums 412 or second pulse maximums 418 to determine the heart rate of the patient 170. The determined heart rate may then be converted into a HRF of the patient 170. That is, peaks of at least one of the first and second BImp measurement signals 404 and 406 are identified and a time between the identified peaks is converted to the heart rate frequency of the patient 170, for example. In some embodiments, the higher stopband frequency of the band-pass filter may be dynamically adjusted based on the determined HRF using peak detection. That is, the higher stopband frequency may be adjusted to a value equal to the HRF plus an additional about 3 Hz. In some embodiments, FFT and peak detection may be combined to determine the HRF of the patient 170.

In some embodiments, peak detection utilises the determined third pulse maximums to determine the heart rate of the patient 170. The determined heart rate may then be converted into a HRF of the patient 170. That is, peaks of the third BImp measurement signal are identified and a time between the identified peaks is converted to the heart rate frequency of the patient 170, for example. In some embodiments, pulse maximums of any one of the BImp measurement signals may be used to determine the heart rate of the patient 170.

At step 208, processor 110 then selects at least one arterial pulse wave feature from the plurality of extracted arterial pulse wave features for each of the first and second BImp measurement signals. In some embodiments, processor 110 selects at least one arterial pulse wave feature from the plurality of extracted arterial pulse wave features for each of the first, second, and third BImp measurement signals. In some embodiments, processor 110 selects at least one arterial pulse wave feature from the plurality of extracted arterial pulse wave features for each of the BImp measurement signals. In some embodiments, processor 110 may select the 1^{st} derivative maximum 408 of the first BImp measurement signal and the 1^{st} derivative maximum 414 of the second BImp measurement signal. For example, processor 110 may select the 1^{st} maximum derivatives 408 and 414, thereby selecting all of the determined 1^{st} derivative maximums 408 and 414 from each heartbeat 422 of the plurality of heartbeats over the measuring time period. Each 1^{st} derivative maximum 408 of the first BImp measurement signal has a corresponding 1^{st} derivative maximum 414 of the second BImp measurement signal for each heartbeat of the plurality of heartbeats of the patient 170, as shown in FIG. 4.

In some embodiments, processor 110 may select the 1^{st} derivative maximum 408 of the first BImp measurement signal, the 1^{st} derivative maximum 414 of the second BImp measurement signal, and the 1^{st} derivative maximum of the third BImp measurement signal. For example, processor 110 may select the 1^{st} maximum derivatives of the first, second, and third BImp measurement signals, thereby selecting all of the determined 1^{st} derivative maximums from each heartbeat of the plurality of heartbeats over the measuring time period. Each 1^{st} derivative maximum 408 of the first BImp measurement signal has a corresponding 1^{st} derivative maximum 414 of the second BImp measurement signal and 1^{st} derivative maximum of the third BImp measurement signal for each heartbeat of the plurality of heartbeats of the patient 170.

In some embodiments, processor 110 may select the 2^{nd} derivative maximum 410 and the 2^{nd} derivative maximums 416. For example, processor 110 may select the 2^{nd} maximum derivatives 410 and 416, thereby selecting all of the determined 2^{nd} derivative maximums 410 and 416 from each heartbeat 422 of the plurality of heartbeats over the measuring time period. In some embodiments, processor 110 may select both the 1^{st} derivative maximums 408 and 414 and the 2^{nd} maximum derivatives 410 and 416. In some embodiments, processor 110 may select the 2^{nd} derivative maximum 410, the 2^{nd} derivative maximums 416, and the 2^{nd} derivative maximum of the third BImp measurement signal. In some embodiments, processor 110 may select both the 1^{st} derivative maximums of the first, second, and third BImp measurement signals and the 2^{nd} maximum derivatives of the first, second, and third BImp measurement signals. In some embodiments, processor 110 may select the 1^{st} derivative maximums and/or the 2^{nd} derivative maximums of each of the BImp measurement signals.

At step 210, processor 110 performing the steps of method 200, determines phase shift information based on the features selected at step 208. In some embodiments, where the processor 110 selected the 1^{st} derivative maximum 408 for the first BImp measurement signal and the 1^{st} derivative maximum 414 of the second BImp measurement signal, phase shift 420 is determined for each 1^{st} maximum derivative 408 and corresponding 1^{st} derivative maximum 414. That is, processor 110 determines a plurality of phase shifts 420 based on the plurality of 1^{st} derivative maximum 408 and their corresponding 1^{st} derivative maximums 414, for example. Each phase shift 420 is determined based on a time difference between the 1^{st} derivative maximum 408 of the first BImp measurement signal 404 and the corresponding 1^{st} derivative maximum 414 of the second BImp measurement signal 406. That is, the phase shift 420 represents the time difference between the 1^{st} derivative maximum 408 and the corresponding 1^{st} derivative maximum 414, for example. In some embodiments, the plurality of phase shifts are based on the plurality of 1^{st} derivative maximums 408, corresponding 1^{st} derivative maximums 414, and corresponding 1^{st} derivative maximums of the third BImp measurement signal. In some embodiments, the plurality of phase shifts are based on the plurality of 1^{st} derivative maximums of each of the BImp measurement signals.

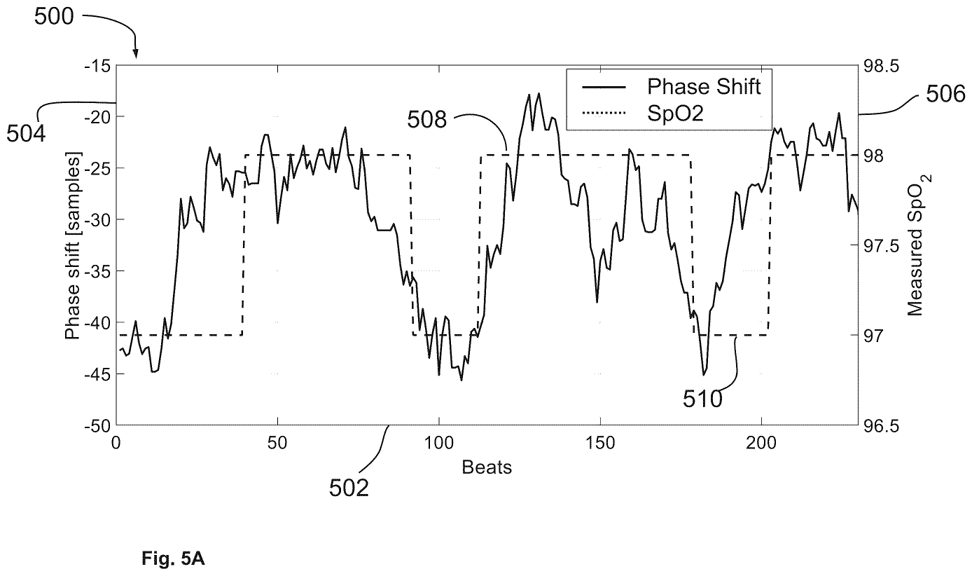

At step 212, processor 110 determines, based on the phase shift information determined at step 210, a blood oxygen saturation (SpO2) percentage of the patient 170 over the measuring time period. That is, the processor 110 determines a SpO2 percentage based on the plurality of phase shifts 420, for example. FIGS. 5A and 5B show example graphs 500 and 501 illustrating the relationship between phase shift and SpO2, according to some embodiments. The x-axis 502 represents a plurality of heartbeats of the patient 170 over at least a portion of the measuring time period. That is, graphs 500 and 501 comprise a data point for each heartbeat 422 of the plurality of heartbeats of the patient 170 measured over at least a portion of the measuring time period, for example. The first y-axis 504 is a measure of the phase shift determined at step 210. The second y-axis 506 is a measure of SpO2 of the patient 170.

Each example graph 500 and 501 comprise a phase shift line graph 508 representing phase shift changes over heartbeats. Each example graph 500 and 501 comprise a SpO2 line graph 510 representing measured SpO2 percentage over heartbeats. Graphs 500 and 501 shows the correlation between phase shifts between the first BImp measurement signal and the second BImp measurement signal and measured SpO2 percentage. Processor 110 determines the SpO2 percentage based on the correlation between phase shifts of two BImp measurement signals. In some embodiments, processor 110 may output the determined SpO2 percentage via user I/O 150. In some embodiments, processor 110 may store the determined SpO2 percentage in memory 130.

In some embodiments, processor 110 determines the SpO2 percentage using a derived mathematical model based on the phase shift information determined at step 210. Processor 110 initially calculates the area under the curve of each of the phase shift line graph 508 and the SpO2 line graph 510. The area under the curve of the phase shift graph 508 may provide information pertaining to key parameters, such as change in phase difference and SpO2, and the duration of the change. In some embodiments, processor 110 calculates the area under the curve of each respective line graph using “Simpsons rule”. Simpsons rule approximates the area of a function by utilising 2^{nd} degree polynomials to model the curve in a selected segment. A selected segment may have a size determined by a time interval. The length of the time interval may be determined by processor 110 or may be predetermined and stored in memory. For example, a selected segment may have a predetermined size of 60 seconds.

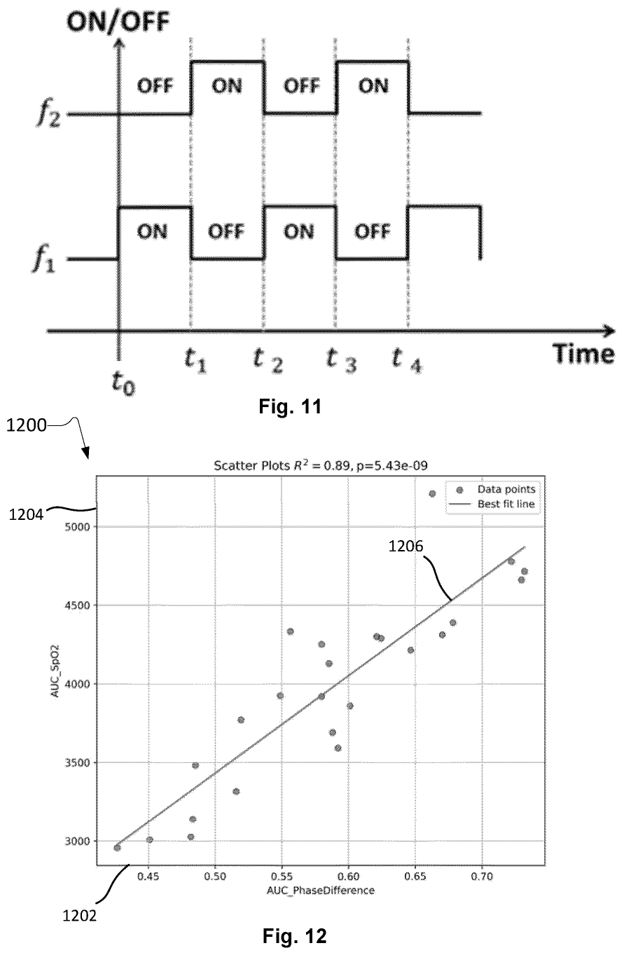

Referring to FIG. 12, there is shown an example graph 1200 illustrating the linear correlation between the area under the curve for each of the phase shift line graph 508 and the SpO2 line graph 510 of example graph 501. Calculated area under the curve of the phase shift line graph 508 is represented on the x-axis 1202 and calculated area under the curve of the SpO2 line graph 510 is represented on the y-axis 1204. A regression analysis is used to determine an R² value and a p-value. The R² value is indicative of the degree to which the data shown in the graph is explained by the determined model. That is, the R² value demonstrates the strength of correlation on a scale of 0 to 1, with a value of 0 meaning no correlation and a value of 1 meaning a high correlation. The p-value represents the statistical significance of the model, where a value lower than 0.05 is indicative of a statistically significant relation between the area under the curve of the phase shift line graph 508 and the area under the curve of the SpO2 line graph 510.

In the example graph 1200 shown in FIG. 12, an R² value of 0.89 was calculated, indicating a high correlation between the two calculated areas under the curve. Additionally, a p-value of 5.42e⁻⁹ was calculated, indicating that there is a statistical significance between the area under the curve of the phase shift line graph 508 and the area under the curve of the SpO2 line graph 510. A line of best fit 1206 is further calculated to determine coefficients, ‘a’ and ‘b’ of the mathematical model for determining SpO2 percentage. Using the determined coefficients, ‘a’ and ‘b’, and the area of the phase difference, represented as ‘A_{pd}’, the area of SpO2, represented as ‘A_{SpO2}’, can be calculated as shown below in equation (1).

$${A}_{Sp O 2} = a \times b \times {A}_{p d} \qquad ( 1 )$$

Each of the calculated A_{SpO2} values are then divided by the selected segment size to calculate an unscaled temporary value corresponding to SpO2. This unscaled temporary value is then scaled to determine the blood oxygen saturation (SpO2) percentage of the patient 170 over the measuring time period. That is, the unscaled derived mathematical model can be represented as shown in equation (2) below. In some embodiments, the derived mathematical model is determined using machine learning and/or artificial intelligence.

$$SpO 2 = \frac{( a \times b \times {A}_{p d} )}{segment size} \qquad ( 2 )$$

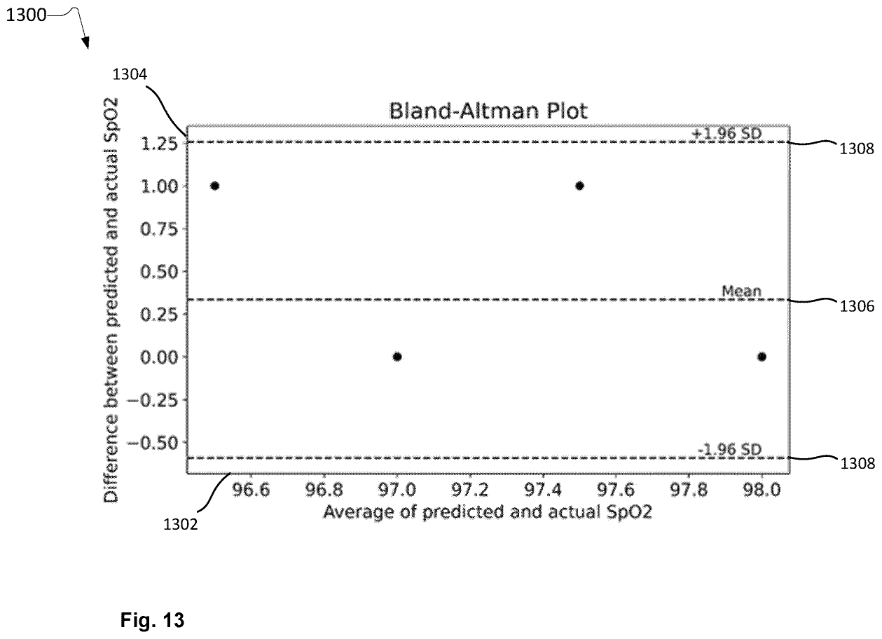

Referring to FIG. 13, there is shown a Bland-Altman plot 1300, comparing SpO2 measured using a known device and SpO2 calculated using the above-described mathematical model. Plot 1300 compares the average of modelled SpO2 and actual measured SpO2 on the x-axis 1302 to the difference between the modelled SpO2 and the actual measured SpO2 on the y-axis 1304. As shown, the data used to form plot 1300 has an average of between about 96% and 98% (x-axis 1302) and a confidence level within 95% as represented by mean 1306 and standard deviations 1308.

In some embodiments, processor 110 may perform method 200 repeatedly and continuously. In some embodiments, processor 110 may perform at least two steps of method 200 synchronously. That is, processor 110 may receive a data stream of BImp measurement signals and perform the steps of method 200 to output a data stream of SpO2 percentage.

In some embodiments, method 200 may be performed by an external computing device. That is, separate computing device from measurement device 100, streams data over Bluetooth or network and then processes it. Previously described the one device doing all of it.

FIG. 6 shows a block diagram of BImp circuit 602, an example embodiment of BImp circuit 160, according to some embodiments. In some embodiments, processor 110, executing measurement code module 132, utilises BImp circuit 602 to receive BImp measurement signals. BImp circuit 602 comprises circuitry 162, sensor 1A 611, sensor 1B 612, sensor 1C 613, and sensor 1D 614. Sensor 1A 611, sensor 1B 612, sensor 1C 613, and sensor 1D 614 are in electrical communication with circuitry 162.

Circuitry 162 comprises a BImp signal generator and processing unit 610 and an analog-to-digital converter (ADC) 620. Sensor 1A 611 is configured to output an electrical signal from a BImp signal generator and processing unit 610 to the patient 170. Sensor 1B 612 is configured to receive an electrical signal from the patient 170 to a BImp signal generator and processing unit 610. That is, sensor 1B 612 is configured to read a first voltage of the electrical signal within the patient 170, for example. Sensor 1C 613 is configured to receive an electrical signal from the patient 170 to a BImp signal generator and processing unit 610. That is, sensor 1C 613 is configured to read a second voltage of the electrical signal within the patient 170, for example. Sensor 1D 614 is configured to receive an electrical signal from the patient 170 to a BImp signal generator and processing unit 610.

The ADC 620 converts an analogue signal received from the BImp signal generator and processing circuit 610 to a digital signal. The ADC 620 is in communication with measurement device 100. That is, measurement device 100 can receive the signal converted by the ADC 620, for example. In some embodiments, the measurement device 100 may store the digital signal received from the ADC 620 in memory 130. In some embodiments, measurement device 100 may process the digital signal received from the ADC 620 by performing method 200 of FIG. 2. That is, measurement device 100 may process the received digital signal from the ADC 620 in real-time, for example. In some embodiments, measurement device 100 receives the digital signal from the ADC 620 at a rate of 1000 samples per second. In some embodiments, measurement device 100 may process the digital signal received from the ADC 620, by performing method 200 of FIG. 2, at a rate of 1000 samples per second.

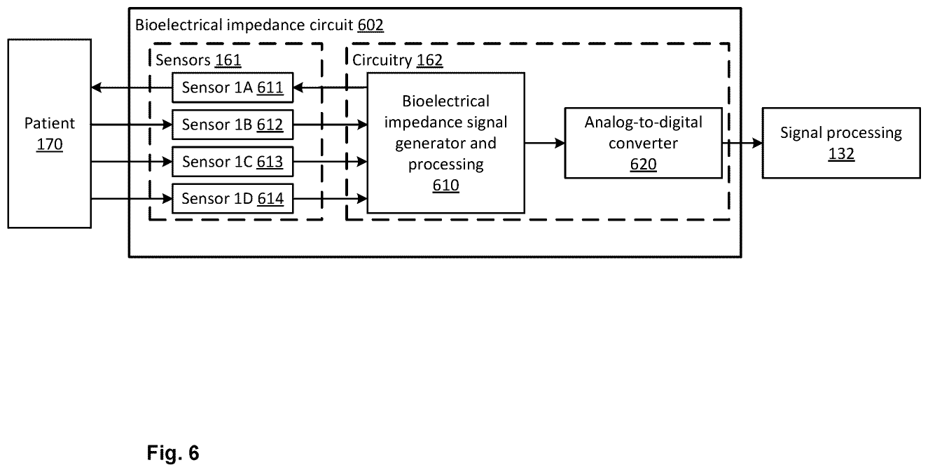

Referring to FIG. 7, an example embodiment of the BImp circuit 602 of FIG. 6 is shown. In some embodiments, the BImp signal generator and processing unit 610 comprises a waveform generation circuit 704, a current limiting circuit 706, a filter circuit 708, a reference electrode 710 and a signal conditioning circuit 712. The waveform generation circuit 704 outputs a clock signal of about 600 mV peak to peak to the sensor 1A 611. In some embodiments, the waveform generation circuit 704 may be a commercially available off the shelf component, such as precision oscillator IC (LTC1799), for example. In some embodiments, the waveform generation circuit 704 further comprises a resistor to set the frequency of the generated clock signal to be injected into the patient via sensor 1A 611. In some embodiments, the resistor may be between about 3KΩ and about 1MΩ). A resistor at about 3KΩ results in a clock signal frequency of about 1 KHz. A resistor at about 1MΩ results in a clock signal frequency of about 33 MHz, for example. The resistance of the waveform generation circuit 704 may be about 91KΩ resulting in a clock signal frequency of about 11 KHz, for example. The resistance of the waveform generation circuit 704 may be about 14KΩ resulting in a clock frequency signal of about 71 KHz, for example. The clock signal frequency may be between about 1 KHz and about 33 MHz, for example.

In some embodiments, the waveform generation circuit 704 and the current limiting circuit 706 may be combined in a commercially available off the shelf component, such as a high precision impedance converter system AD5933 or AD5941. In said embodiments, the processor 110 determines the shape and frequency of the injected signal and limits the output current based on data values stored in memory 130.

In some embodiments, the waveform generation circuit 704 further comprises a low-pass filter to alter the shape of the generated clock signal. The low-pass filter of the waveform generation circuit 704 may further include a tuneable cut-off frequency. In some embodiments, the shape of the altered generated clock signal is sinusoidal. The sinusoidal clock signal may determine the voltage source of the waveform generation circuit 704. The current limiting circuit 706 may comprise at least one resistor to limit the current output by the waveform generation circuit 704 to the sensor 1A 611. In some embodiments, the at least one resistor may be between about 1KΩ and about 5KΩ. A resistor value of about 5KΩ may produce a current output of about 400 μA, for example. The current limiting circuit 706 further comprises a DC blocking capacitor to prevent output of DC current by the waveform generation circuit 704 to the patient 170 via sensor 1A 611.

The filter circuit 708 is an analogue filter circuit comprising a differential RC band-pass filter. The filter circuit 708 may have a higher cut-off frequency of about 100 Hz, for example. The filter circuit 708 may have a lower cut-off frequency of about 0.16 Hz, for example. The filter circuit 708 may have a gain of about 0.5, for example. The signal conditioning circuit 712 extracts the raw impedance waveform received via the sensor 1B 612. The signal conditioning circuit 712 extracts the raw impedance waveform received via the sensor 1C 613. The signal conditioning circuit 712 comprises an instrumentation amplifier IC and a low-pass filter circuit. The instrumentation amplifier IC may have a gain of about 100. The low-pass filter of the signal conditioning circuit 712 may further include a tuneable cut-off frequency. The cut-off frequency of the low-pass filter of the signal conditioning circuit may be about 20 Hz, for example. The signal conditioning circuit 712 provides an analogue signal to the ADC 620.

The reference electrode 710 acts to ensure that current injected via sensor 1A 611 is directed away from sensor 1B 612 and sensor 1C 613. That is, the reference electrode 710, in communication with sensor 1D 614, ensures stray current on the surface of the skin of the patient 170 is directed away from sensor 1B 612 and sensor 1C 613, for example.

In some embodiments, processor 110 performs time-multiplexing with BImp signal generator and processing unit 710 to generate a first signal of a first frequency and a second signal of a second frequency for injection into the patient via sensor 1A 611. That is, processor 110 switches the frequency of the signal generated by the waveform generation circuit 704 for each sample provided to the ADC 620. For example, the total sample rate of the ADC 620 is 1000 samples per second, resulting in 500 samples of the first signal of the first frequency and 500 samples of the second signal of the second frequency per second. The processor 110 configures the waveform generation circuit 704 to output the first signal at the first frequency for one sample of the 1000 samples. The processor 110 then configures the waveform generation circuit 704 to output the second signal at the second frequency for next sample of the 1000 samples. The processer 110 then configures the waveform generation circuit 704 to again output the first signal of the first frequency for the next sample of the 1000 samples. This is repeated until measurement code module 132 is no longer being executed by processor 110.

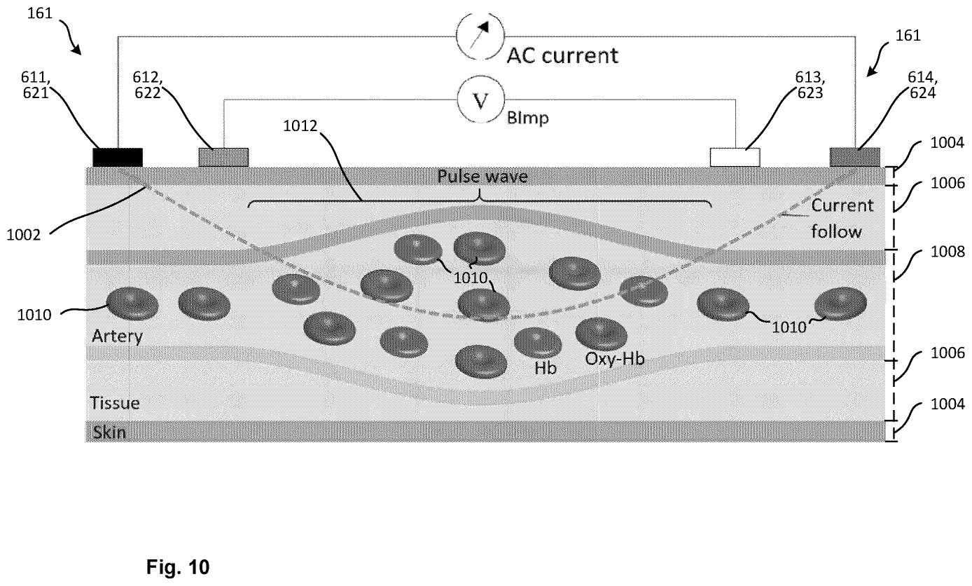

Referring to FIG. 11, there is shown an example frequency switching timing diagram for switching between the first signal of the first frequency, f1, and the second signal of the second frequency, f2, according to some embodiments. That is, FIG. 11 shows an example frequency switching timing diagram for performing time-multiplexing with BImp signal generator and processing unit 710, for example. Processor 110 configures or causes the waveform generation circuit 704 to output the first signal at the first frequency for time t₀-t₁ interval. That is, at time to, the first signal is considered “on” and the second signal is considered “off”, Processor 110 then configures or causes the waveform generation circuit 704 to output the second signal at the second frequency for time t₁-t₂ and to cease outputting the first signal. That is, at time t₁, the first signal is considered “off” and the second signal is considered “on”. Processor 110 then configures or causes the waveform generation circuit 704 to again output the first signal at the first frequency for time t₂-t₃ and to cease outputting the second signal. That is, at time t₂, the first signal is again considered “on” and the second signal is again considered “off”, Processor 110 repeats the switching of the first and second signals in this manner until the functions of measurement code module 132 are no longer being executed by processor 110.

The time intervals between the time points of t₀, t₁, t₂, t₃, and t₄ may range between about 250 us (microseconds) and about 30 ms, for example. The time intervals between t₀, t₁, t₂, t₃, and t₄ each have the same time length according to some embodiments. The time intervals between the time points of t₀, t₁, t₂, t₃, and t₄ may be selected dependent on the required number of samples and/or the total sample rate of the ADC 620. For example, a minimum required number of samples may be at least 200 samples per second, and at an interval of 30 ms for intervals between t₀, t₁, t₂, t₃, and t₄, the ADC 620 receives the at least 200 samples per second. In some embodiments, the ADC 620 is configured to receive a particular number of samples per second irrespective of the time interval. That is, the waveform generation circuit 704 may output the first signal at the first frequency and the second signal at the second frequency such that 1000 samples may be received by the ADC 620, however, the ADC 620 may only receive 200 samples of the 1000 samples, for example.

In some embodiments, the length of the time intervals between t₀, t₁, t₂, t₃, and t₄ may be dependent on the heart rate frequency of the patient 170. That is, a higher heart rate frequency (HRF) may require an increased resolution to measure shorter pulse durations, therefore, the length of the time intervals t₁, t₂, t₃, and t₄ may be reduced to a lower value when the HRF increases, for example. Similarly, the length of the time intervals between t₀, t₁, t₂, t₃, and t₄ may be increased to a higher value when the HRF decreases, for example.

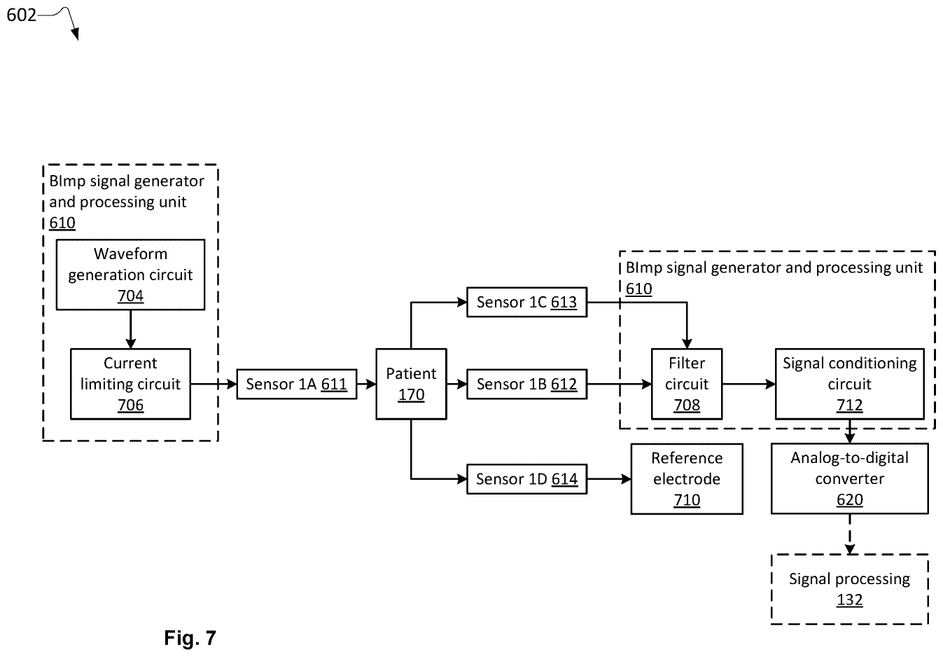

FIG. 8 shows a block diagram of BImp circuit 802, an alternate example embodiment of BImp circuit 160, according to some embodiments. In some embodiments, processor 110, executing measurement code module 132, utilises BImp circuit 802 to receive BImp measurement signals. BImp circuit 802 is a further instance of BImp circuit 602 and further comprising circuitry 162, sensor 2A 811, sensor 2B 812, sensor 2C 813, and sensor 2D 814. Sensor 2A 811, sensor 2B 812, sensor 2C 813, and sensor 2D 814 are in electrical communication with circuitry 162. Circuitry 162 of BImp circuit 802 is a further instance of circuitry 162 of BImp circuit 602 further comprising BImp signal generator and processing unit 810. Sensor 2A 811 is configured to output an electrical signal from BImp signal generator and processing unit 810 to the patient 170. Sensor 2B 812 is configured to receive an electrical signal from the patient 170 to BImp signal generator and processing unit 810. Sensor 2C 813 is configured to receive an electrical signal from the patient 170 to BImp signal generator and processing unit 810. Sensor 2D 814 is configured to receive an electrical signal from the patient 170 to BImp signal generator and processing unit 810.

The ADC 620 converts an analogue signal received from the BImp signal generator and processing circuit 810 to a digital signal. Measurement device 100 may receive the converted BImp signal generator and processing circuit 810 signal as previously described in relation to the BImp signal generator and processing circuit 610 of FIG. 6.

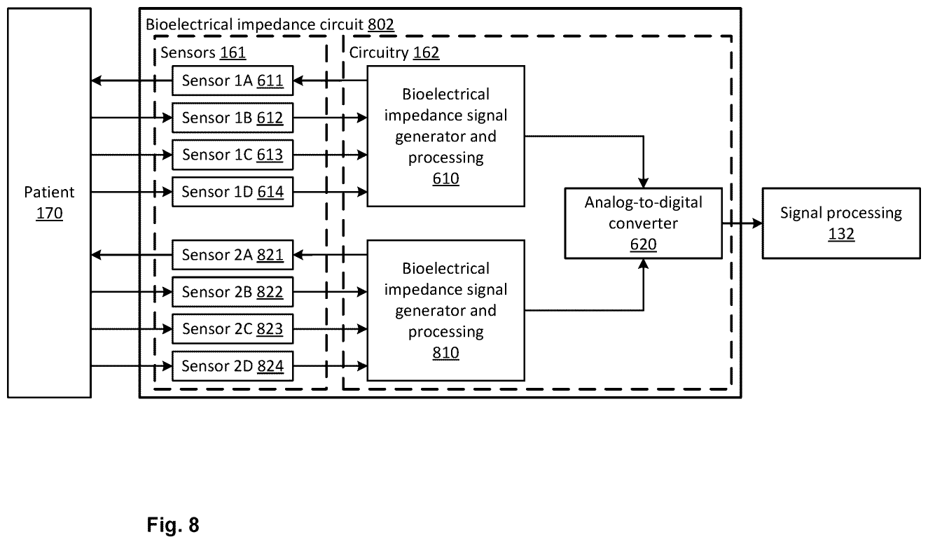

Referring to FIG. 9, an example embodiment of the BImp circuit 802 of FIG. 8 is shown. BImp signal generator and processing unit 810 is a further instance of BImp signal generator and processing unit 610, wherein the BImp signal generator and processing unit 810 and BImp signal generator and processing unit 610 produce two sinusoidal waveforms of different frequencies. That is, the BImp signal generator and processing unit 610 may generate a first signal of a first frequency and the BImp signal generator and processing unit 810 may generate a second signal of a second frequency for injection to the patient via sensor 1A 611 and sensor 2A 621, respectively, for example. The resistance of the waveform generation circuit 704 of the BImp signal generator and processing unit 610 may be about 91KΩ, resulting in a clock frequency of the first signal of about 11 KHz, for example. The resistance of the waveform generation circuit 704 the BImp signal generator and processing unit 810 may be about 14KΩ resulting in a clock frequency of the second signal of about 71 KHz, for example.

The BImp signal generator and processing unit 610 comprises a first waveform generation circuit 704 and the BImp signal generator and processing unit 610 comprises a second waveform generation circuit 704. In some embodiments, the first and second waveform generation circuits 704 and their respective current limiting circuits 706, of each of the BImp signal generator and processing unit 610 and 810, may be combined in a commercially available off the shelf component, such as an high precision impedance converter system AD5933 or AD5941. In said embodiments, the processor 110 determines the shape and frequency of the injected first and second signals and limits the output current of each signal based on data values stored in memory 130.

In some embodiments, each of the first and second waveform generation circuits 704 further comprises a low-pass filter to alter the shape of the generated first and second clock signals. The low-pass filter of each of the first and second waveform generation circuits 704 may further include a tuneable cut-off frequency. In some embodiments, the shape of the altered generated first and second clock signals is sinusoidal. The first and second sinusoidal clock signals may determine the voltage source of each of the first and second waveform generation circuits 704, respectively. The current limiting circuit 706 of each of the BImp signal generator and processing units 610 and 810 may comprise at least one resistor to limit the current output by each of the first and second waveform generation circuits 704 to the sensor 1A 611 and sensor 2A 621, respectively. In some embodiments, the at least one resistor may be between about 1KΩ and about 5KΩ. A resistor value of about 5KΩ may produce a current output of about 400 μA, for example. Each current limiting circuit 706 further comprises a DC blocking capacitor to prevent output of DC current by the waveform generation circuit 704 to the patient 170 via sensor 1A 611 and sensor 2A 621.

The filter circuit 708 of each of the BImp signal generator and processing units 610 and 810 is an analogue filter circuit comprising a differential RC band-pass filter. Each filter circuit 708 may have a higher cut-off frequency of about 100 Hz, for example. Each filter circuit 708 may have a lower cut-off frequency of about 0.16 Hz, for example. Each filter circuit 708 may have a gain of about 0.5, for example. The signal conditioning circuit 712 of each of the BImp signal generator and processing units 610 and 810 extracts the raw impedance waveform received via the sensor 1B 612 and the sensor 2B 622, respectively. Each signal conditioning circuit 712 extracts the raw impedance waveform received via the sensor 1C 613 and the sensor 2C 623. Each signal conditioning circuit 712 comprises an instrumentation amplifier IC and a low-pass filter circuit. Each instrumentation amplifier IC may have a gain of about 100. Each low-pass filter of each signal conditioning circuit 712 may further include a tuneable cut-off frequency. The cut-off frequency of the low-pass filter of each signal conditioning circuit may be about 20 Hz, for example. Each signal conditioning circuit 712 provides an analogue signal to the ADC 620.

The reference electrode 710 acts to ensure that current injected via sensor 1A 611 and sensor 2A 621 is directed away from sensors 1B 612 and 1C 613 and sensors 2B 622 and 2C 623, respectively. That is, the reference electrode 710, in communication with sensor 1D 614 and sensor 2D 624, ensures stray current on the surface of the skin of the patient 170 is directed away from sensor 1B 612 and 1C 613 and sensors 2B 622 and 2C 623, respectively, for example.

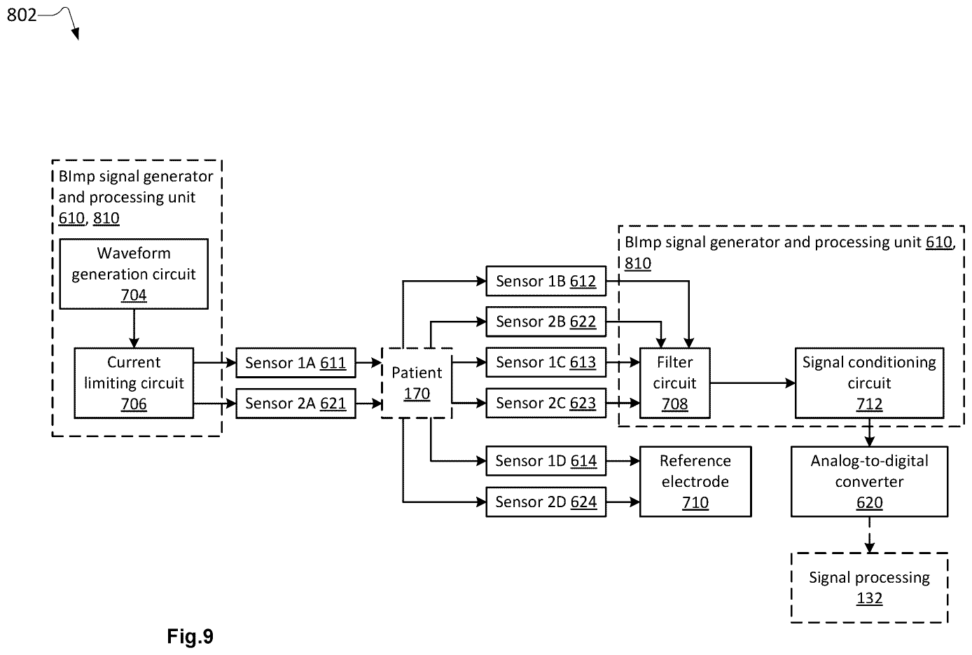

FIG. 10 is an illustration showing measurement of a signal injected through an artery of a patient 170. As shown in FIG. 10, there is an artery 1008 in which haemoglobin 1010 (oxyhaemoglobin and deoxyhaemoglobin) travels. The artery 1008 is surrounded at least in part by a portion of tissue 1006, the tissue being surrounded in part by a layer of skin 1004. In electrical communication with the skin 1004 of the patient 170 are sensors 161. In embodiments as described in relation to FIGS. 6 and 7, sensors 1A 611, sensors 1B 612, sensors 1C 613, and sensors 1D 614 are included. In said embodiments, a first signal of a first frequency is injected via sensor 1A 611 along the current flow path 1002 to sensor 1D 614. Sensor 1B 612 measures, or reads, the voltage of the injected first signal at a first location of a pulse wave 1012. Sensor 1C 613 measures, or reads, the voltage of the injected first signal at a second location of the pulse wave 1012. The difference between the measurements of sensor 1B 612 and sensor 1C 613 of the injected first signal is used to determine a first BImp measurement signal. That is, the difference between the measurements of sensor 1B 612 and sensor 1C 613 is a measure of the changing impedance of haemoglobin within the artery of the patient 170 over time, for example.

In embodiments utilising time-multiplexing, a second signal of a second frequency is injected via sensor 1A 611 along the current flow path 1002 to sensor 1D 614. Sensor 1B 612 measures, or reads, the voltage of the injected second signal at the first location of the pulse wave 1012. Sensor IC 613 measures, or reads, the voltage of the injected second signal at the second location of the pulse wave 1012. The difference between the measurements of sensor 1B 612 and sensor IC 613 of the injected second signal is used to determine a second BImp measurement signal.

In embodiments as described in relation to FIGS. 8 and 9, sensors 2A 621, sensors 2B 622, sensors 2C 623, and sensors 2D 624 are further included. In said embodiments, the first signal of the first frequency is injected via sensor 1A 611 along the current flow path 1002 to sensor 1D 614. The second signal of the second frequency is injected via sensor 2A 621 along the current flow path 1002 to sensor 2D 624. Sensor 1B 612 measures, or reads, the voltage of the injected first signal at the first location of the pulse wave 1012. Sensor 1C 613 measures, or reads, the voltage of the injected first signal at the second location of the pulse wave 1012. The difference between the measurements of sensor 1B 612 and sensor 1C 613 of the injected first signal is used to determine the first BImp measurement signal. Sensor 2B 622 measures, or reads, the voltage of the injected second signal at the first location of the pulse wave 1012. Sensor 2C 623 measures, or reads, the voltage of the injected second signal at the second location of the pulse wave 1012. The difference between the measurements of sensor 2B 622 and sensor 2C 623 of the injected second signal is used to determine the second BImp measurement signal.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method of determining blood oxygen saturation, comprising:
receiving, at a computing device, a first bioimpedance (BImp) measurement signal of a patient, wherein the first BImp measurement signal is of a first frequency;
receiving, at a computing device, a second bioimpedance measurement signal of a patient, wherein the second BImp measurement signal is of a second frequency different from the first frequency;
wherein, including, for each of the first and second BImp measurement signals, an arterial pulse wave representing impedance changes through an artery of the patient over time;
filtering each of the first and second BImp measurement signals using a band-pass filter, wherein the band-pass filter defines a frequency band;
performing, on the first and second filtered BImp measurement signals, feature point extraction to generate a plurality of arterial pulse wave features;
selecting, from the plurality of arterial pulse wave features, at least one arterial pulse wave feature for each of the first and second BImp measurement signals;
comparing the at least one selected feature of the first BImp measurement signal and the at least one selected feature of the second BImp signal to determine phase shift information, wherein the phase shift information represents a phase shift between the first and second BImp measurement signals; and
determining, based on the determined phase shift information, a blood oxygen level and saturation percentage of the patient.

2. The method of claim 1, wherein the first and second BImp measurement signals are received from a sensing device worn by the patient.

3. The method of claim 1, wherein the first frequency is in the range of about 5 KHz to about less than 50 KHz.

4. The method of claim 1, wherein the second frequency is in the range of about more than or equal to 50 KHz to about 100 KHz.

5. The method of claim 1, wherein the receiving is performed continuously and wherein the steps of filtering, performing, selecting, comparing, and determining are performed repeatedly while receiving occurs.

6. The method of claim 1, wherein the frequency band of the band-pass filter has a lower stopband frequency between about 0.5 Hz and about 0.95 Hz.

7. The method of claim 1, wherein the frequency band of the band-pass filter has a higher stopband frequency between about 15 Hz and about 20 Hz.

8. The method of claim 1, further comprising determining, based on at least one of the first and second BImp measurement signals, a heart rate frequency of the patient.

9. (canceled)

10. (canceled)

11. (canceled)

12. A method of diagnosing a medical condition including performing the method of claim 1.

13. The method of claim 1, further comprising, determining, based on the determined phase shift information, a mathematical model for determining a blood oxygen level and saturation percentage of the patient, and subsequently determining the blood oxygen level and saturation percentage of the patient using the mathematical model.

14. A computing device for monitoring a blood condition, comprising:
processing circuitry;
a memory accessible to the processing circuitry, the memory including a signal processing code module;
a communications module accessible to the processing circuitry, wherein the signal processing code module includes instructions, executable by the processing circuitry, to process bioimpedance (BImp) measurement signals received via the communications module; and
wherein the signal processing code module further includes instructions, executable by the processing circuitry, to perform the following:
filter at least two BImp measurement signals of different frequencies using a band-pass filter, wherein the band-pass filter defines a frequency band;
perform feature point extraction on the at least two filtered BImp measurement signals to generate a plurality of arterial pulse wave features;
select, from the plurality of arterial pulse wave features, at least one arterial pulse wave feature for each of the at least two BImp measurement signals;
compare the at least one selected feature of each of the at least two BImp measurement signals to determine phase shift information, wherein the phase shift information represents a phase shift between the at least two BImp measurement signals; and
determine, based on the determined phase shift information, an oxygen saturation percentage of the patient.

15. The computing device of claim 14, wherein the at least two BImp measurement signals are of different frequencies.

16. The computing device of claim 14, wherein the signal processing code module further includes instructions to determine, based on at least one of the at least two transformed BImp measurement signals, a heart rate frequency of the patient.

17. The computing device of claim 14, wherein the instructions of the signal processing code module are performed continuously.

18. (canceled)

19. The computing device of claim 14, wherein the frequency band of the band-pass filter has a lower stopband frequency between about 0.5 Hz and about 0.95 Hz and a higher stopband frequency between about 15 Hz and about 20 Hz.

20. A system including the computing device of claim 14, and further including, a wearable device, wherein the wearable device comprises:
processing circuitry;
a memory accessible to the processing circuitry, the memory including a measurement code module;
a communications module accessible to the processing circuitry, wherein the measurement code module includes instructions, executable by the processing circuit, to transmit bioimpedance (BImp) measurement signals to the computing device via the communications module;
at least two electrodes attachable to skin of a person; and
wherein the measurement code module further includes instructions, executable by the processing circuitry, to perform the following:
output, via at least one of the at least two electrodes, at least two electrical stimulation signals to a patient, wherein the at least two electrical stimulation signals are of different frequencies;
detect, via at least one of the at least two electrodes, at least two BImp measurement signals, wherein the at least two BImp measurement signals are based on the at least two electrical stimulation signals; and
transmit, via the communications module, the at least two detected BImp measurement signals to the computing device.

21. The system of claim 20, wherein the measurement code module is further configured to output the at least two electrical stimulation signals using time-multiplexing.

22. (canceled)

23. A kit including the computing device of claim 14, and further including a wearable device, wherein the wearable device comprises:
processing circuitry;
a memory accessible to the processing circuitry, the memory including a measurement code module;
a communications module accessible to the processing circuitry, wherein the measurement code module includes instructions, executable by the processing circuit, to transmit bioimpedance (BImp) measurement signals;
at least two electrodes attachable to skin of a person; and
wherein the measurement code module further includes instructions, executable by the processing circuitry, to perform the following:
output, via at least one of the at least two electrodes, at least two electrical stimulation signals to a patient, wherein the at least two electrical stimulation signals are of different frequencies;
detect, via at least one of the at least two electrodes, at least two BImp measurement signals, wherein the at least two BImp measurement signals are based on the at least two electrical stimulation signals;
transmit, via the communications module, the at least two detected BImp measurement signals to the computing device; and
wherein the computing device is configured to receive BImp measurement signals from the wearable device.

24. A method of determining blood oxygen saturation, comprising:
receiving, at a computing device, at least two bioimpedance (BImp) measurement signals of a patient, the at least two BImp measurement signals include a first BImp measurement signal and a second BImp measurement signal, wherein the first BImp measurement signal is of a first frequency and the second BImp measurement signal is of a second frequency different from the first frequency;
wherein, including, for each of the at least two BImp measurement signals, an arterial pulse wave representing impedance changes through a vascular bed of the patient over time;
filtering each of the at least two BImp measurement signals using a band-pass filter, wherein the band-pass filter defines a frequency band;
performing, on the at least two filtered BImp measurement signals, feature point extraction to generate a plurality of arterial pulse wave features;
selecting, from the plurality of arterial pulse wave features, at least one arterial pulse wave feature for each of the at least two BImp measurement signals;
comparing the selected features of the at least two BImp measurement signals to determine phase shift information, wherein the phase shift information represents a phase shift between the at least two BImp measurement signals; and
determining, based on the determined phase shift information, a blood oxygen level and saturation percentage of the patient.

25. (canceled)

26. The method of claim 24, wherein the at least two BImp measurement signals include a plurality of BImp measurement signals, wherein each of the plurality of BImp measurement signals is of a different frequency.
